(19) United States
(12) Patent Application Publication
(10) Pub. No.: US 2003/0157556 A1
(43) Pub. Date: Aug. 21, 2003

(54) Biological fluid stabilizing composition and method of use thereof

(76) Inventors: Jack A. Maggiore, Lombard, IL (US); Barbara R. Grzeda, Schaumburg, IL (US)

Correspondence Address: OLSON & HIERL, LTD., 36th Floor, 20 North Wacker Drive, Chicago, IL, 60606, US

(21) Appl. No.: 10/074,715

(22) Filed: Feb. 13, 2002

(51) Int. Cl. A01N 1/02; C12Q 1/68; G01N 33/53

(52) U.S. Cl. 435/7.1; 435/2; 435/6

(57) ABSTRACT

An aqueous biological fluid preserving composition consists essentially of a chelating agent, such as EDTA, and a cell lysing amount of a cell lysing agent, such as an alcohol. The biological fluid preserving composition can also include a preservative, an antifreeze agent and a non-chelating anticoagulant. The aqueous biological fluid preserving composition is useful for preserving biological fluids collected for later quantitative determination of protein components such as hormones.

### FIELD OF THE INVENTION

This invention relates to compositions and methods for stabilizing biological fluids. More particularly, this invention relates to compositions and methods for stabilizing biological fluid samples for analysis of protein analytes.

### BACKGROUND OF THE INVENTION

Many conditions and disease states can be diagnosed by quantitative analysis of biological fluid specimens for chemical markers indicative of the particular condition or disease state. Many analytes of medical interest are proteins, which can often be difficult to handle and store for later analysis due to the tendency of proteins to denature. Quite often, the difficulty of handling and preparing biological fluid samples for analysis necessitates that the patient must visit a laboratory, doctor's office or other out-patient facility in order to collect the specimen for analysis.

Recently, at-home collection systems have been developed for collecting biological fluid specimens for delivery to a testing laboratory for later analysis. Such systems are generally utilized for easily preservable biological fluid specimens where the analyte is stable, or can be easily stabilized, such as a system designed for analysis of cholesterol in blood, marketed by Biosafe Medical Technologies, Inc. of Lake Forest, Ill. Analysis of protein-based hormones in biological fluid samples, such as thyroid stimulating hormone in blood, can be particularly challenging due to the delicate nature of the analyte and the need to lyse the cellular components of the specimen prior to analysis.

There is an ongoing need, therefore, for a biological fluid preserving composition that will preserve protein analytes under conditions that allow home collection of the biological fluid specimen and subsequent delivery of the preserved specimen to a clinical laboratory for analysis without significant degradation of the analyte. The aqueous biological fluid preserving compositions of the present invention fulfill this need

### SUMMARY OF THE INVENTION

Biological fluid specimens are preserved and stabilized for later analysis by admixing the specimen with an aqueous biological fluid preserving composition containing a chelating agent and a cell lysing amount of a cell lysing agent. The aqueous composition can also include additional additives and adjuvants such as preservatives, antifreeze agents and non-chelating anticoagulants.

The biological fluid preserving compositions of the present invention advantageously rapidly lyse cellular components of biological fluids while stabilizing protein analytes, such as hormones, which are present in the fluid specimen. The inventive aqueous preservative compositions are particularly useful for preserving samples of biological fluids containing protein analytes that must be stored or transported for a period of time prior to quantitative determination of the protein and other analytes in the specimen.

In one preferred embodiment, a biological fluid preservative composition consists essentially of a cell lysing amount of a C₁- C₄ alcohol and a calcium-chelating agent in water. The aqueous biological fluid preserving compositions of the present invention can preserve protein-containing biological fluid specimens, generally for periods of up to about a week or more, in a condition suitable for quantitative determination of protein analytes within the biological fluid specimen by a variety of standard protein analytical methods.

The aqueous biological fluid preserving compositions of the present invention are particularly suitable for preservation and hemolysis of blood samples for later quantitative analysis for hormones such as thyroid stimulating hormone (TSH), follicle stimulating hormone (FSH), luteinizing hormone (LH), human chorionic gonadotropin (HCG), and the like.

### BRIEF DESCRIPTION OF THE DRAWINGS

In the Drawings, FIG. 1 is a graphical representation of the stability of low blood concentration levels of TSH from whole blood preserved in a biological fluid preserving composition of the invention, at temperatures in the range of about 2 to about 45° C. for periods of about 7 to about 21 days;

FIG. 2 is a graphical representation of the stability of medium blood level concentrations of TSH from whole blood preserved in a biological fluid preserving composition of the invention, at temperatures in the range of about 2 to about 45° C. for periods of about 7 to about 21 days; and

FIG. 3 is a graphical representation of the stability of high blood levels of TSH from whole blood preserved in a biological fluid preserving composition of the invention, at temperatures in the range of about 2 to about 45° C. for periods of about 7 to about 21 days.

### DETAILED DESCRIPTION OF PREFERRED EMBODIMENTS

As used herein, the term “cell lysing agent”, and grammatical variations thereof, means any chemical medium or substance capable of disrupting the cellular membrane of cells in contact with the medium or substance, causing the membrane to rupture and thus releasing the contents of the cell.

The term “chelating agent” and grammatical variations thereof, as use herein” means any chemical substance capable of chelating, complexing, or sequestering alkaline earth or transition metal ions. The term chelating agent includes complexing agents and sequestering agents.

As used herein, the term “preservative” and any grammatical variation thereof means a chemical agent that can prevent or delay the degradation or decay of organic substances such as biological fluid specimens. The term preservatives includes, without being limited thereto, biocides, such as anti-microbial agents, fungicides, and anti bacterial agents; antioxidants; disinfectants; antiseptics; and the like. As used herein, the term “antifreeze agent” means any water soluble substance that will lower the freezing point of an aqueous solution.

Biological fluid specimens are preserved and stabilized for later analysis by admixing the specimen with an aqueous composition consisting essentially of a chelating agent and a cell lysing amount of a cell lysing agent in water. The aqueous composition can also include additional additives and adjuvants such as preservatives, antifreeze agents, pH adjusting agents, surfactants, and the like. In compositions for the preservation of whole blood specimens, non-chelating anticoagulants can also be advantageously included in the composition.

Biological fluid specimens that can be preserved by the compositions of the present invention include, but are not limited to whole blood, plasma, serum, saliva, semen, milk, urine, and amniotic fluid. The compositions of the present invention are particularly useful for the stabilization and preservation of protein analytes in the biological fluids. The aqueous preservative compositions of the invention are particularly useful for preserving samples of biological fluids that must be stored for a period of time prior to quantitative determination of various protein components present in the fluids.

The biological fluid preserving compositions of the present invention advantageously rapidly lyse cellular components of biological fluids while stabilizing protein analytes that are present in the fluid specimen, and are substantially free of formalin.

In one preferred embodiment, an aqueous biological fluid preserving composition consists essentially of a cell lysing amount of a C₁- C₄ alcohol and a calcium-chelating agent in water.

In another preferred embodiment, an aqueous biological fluid preserving composition consists essentially of a chelating agent, a cell lysing amount of a cell lysing agent, a preservative, and optionally an anti-freeze agent, in water.

In a particularly preferred embodiment, the biological fluid preserving composition consists essentially of, on a total composition weight basis, about 5 to about 25 weight percent of a cell lysing agent, about 0.05 to about 0.5 weight percent of a chelating agent, and up to about 0.1 weight percent of a preservative, the remainder being water. The biological fluid preserving composition preferably also includes about 0.01 to about 0.03 weight percent of a preservative. Optionally, the biological fluid preserving composition can include up to about 50 weight percent of an anti-freeze agent, preferably about 43 to about 49 weight percent.

Cell lysing agents useful in the compositions of the present invention include, but are not limited to, water soluble organic solvents such as alcohols, ethers, ketones, polar aprotic solvents, and the like. Preferred cell lysing agents include C₁- C₄ alcohols. Preferred C₁- C₄ alcohols include methanol, ethanol and isopropanol. A particularly preferred cell lysing agent is ethanol.

The aqueous biological fluid preserving composition of the present invention preferably includes about 5 to about 25 weight percent of a cell lysing agent, more preferably about 5 to about 15 weight, and most preferably about 6 to about 10 weight percent, on a total composition weight basis.

The cell lysing agents useful in the compositions of the present invention can be general cell lysing agents, such as organic solvents, or hemolytic cell lysing agents, such as hemolysins. Hemolysins include, but are not limited to, bacterial hemolysins, acid hemolysins, hemolytic antibodies, and the like.

Chelating agents suitable for use in the compositions of the present invention include, without being limited to, polyphosphates, amino carboxylic acids, 1,2-diketones, hydroxycarboxylic acids, polyamines, amino alcohols, aromatic heterocyclic bases, phenols, aminophenols, oximes, Schiff bases, tetrapyrroles, thiols, xanthates, polycarboxylic acids, polyphosphonic acids, polysulfonic acids, and the like, and salts thereof.

Preferred chelating agents include ethylenediaminetetracetic acid (EDTA), nitrilotriacetic acid (NTA), amino-tris-(methylenephosponic acid), hydroxyethylidene diphosphonic acid, polyacrylic acid, polymaleic acid, acrylic acid/maleic acid copolymer, gluconic acid, citric acid, oxalic acid, and the like, and salts thereof. Preferably the chelating agents are calcium chelating agents. Preferred salts of chelating agents include alkali metal salts such as lithium, sodium and potassium salts; as well as ammonium salts.

Chelating, complexing, and sequestering agents are well known in the chemical art and are commercially available from a variety of sources. Commercial chelating, complexing, and sequestering agents and commercial sources thereof are listed in McCutcheon's, Vol. 2, Functional Materials, McCutcheon's Division, The Manufacturing Confectioner Publishing Co., Glenrock, N.J. (2001), the relevant disclosures of which are incorporated herein by reference.

Preservatives useful in the aqueous biological fluid preserving compositions of the present invention include antimicrobial agents and antioxidants, but exclude compounds and materials that protect the cell structure from lysis, e.g. formaldehyde, formalin solutions, and the like. Examples of anti-microbial agents useful in the compositions of the present invention include, without being limited to, quaternary ammonium compounds, isothiazolinones, thiocarbamates, aldehydes, halo-organic compounds, organic nitro compounds, phenols, azides, and the like.

Suitable antioxidants include water soluble antioxidants, such as butylated hydroxy toluene (BHT), butylated hydroxy anisole (BHA), ascorbic acid, inorganic hypophosphites such as hypophosphorus acid and sodium hypophosphite; and the like.

Preferred preservatives include sodium azide, isothiazolinones, glutaraldehyde, chlorophenols, BHA, BHT, 2-bromo-2-nitropropane-1,3-diol, sodium dimethyldithiocarbamate, and combinations thereof. A particularly preferred preservative is sodium azide.

Antifreeze agents that are useful in the compositions of the present invention are preferably water soluble organic polyols such as glycols, gylcol oligomers, glycerin, and the like. Preferred antifreeze agents are water soluble C₁-C₁₀ organic polyols. Particularly preferred polyol-based antifreeze agents include ethylene glycol, propylene glycol, butylene glycol, hexylene glycol diethylene glycol, triethylene glycol, tetraethylene glycol, glycerin, hexane-1,6-diol, inositol, and combinations thereof.

When desired, antifreeze agents can be included in the aqueous biological fluid preserving compositions of the present invention at a concentration of up to about 50 weight percent of the composition, on a total composition weight basis, preferably about 25 to about 50 weight percent, more preferably about 43 to about 49 weight percent.

The aqueous biological fluid preserving compositions of the present invention can preserve protein-containing biological fluid specimens, generally for periods of up to about two weeks or more, in a condition suitable for quantitative determination of protein analytes within the biological fluid specimen by a variety of standard protein analytical methods.

The aqueous biological fluid preservative compositions of the present invention are particularly suitable for preservation and hemolysis of blood samples for later quantitative analysis for hormones such as TSH, FSH, LH, HCG, and the like.

In use, a specimen of a biological fluid is combined and diluted with preferably about 0.5 to about 8 volumes of the inventive biological fluid preserving composition, based on the volume of the biological fluid specimen. More preferably, the specimen is diluted with about 1 to about 5 volumes of the preservative composition, most preferably about 2 to about 4 volumes.

The so-diluted specimen is then preferably sealed in a suitable container, such as a glass or plastic vial, tube, bottle, or special purpose biological fluid sample collection vessel such as the BTS™ Biosafe Blood Transportation System marketed by Biosafe Medical Technologies, Inc. of Lake Forest, Ill. The filled, sealed container can then be packaged in a suitable transportation container and shipped to a clinical analytical laboratory for analysis of the specimen. Dilution of the specimen with a biological fluid preserving composition of the present invention stabilizes and preserves protein analytes present in the specimen during transport, while lysing the cellular components of the specimen.

The biological fluid preserving compositions of the present invention can preserve a specimen of a biological fluid, such as a whole blood specimen, when stored for up to about one week at a temperature of about 45° C., and at least about 3 weeks at ambient room temperature or below. Such a preserved specimen can be analyzed for specific protein analytes, such as TSH, without any significant loss in the measurable protein concentration after the storage or transport period is ended. When the biological fluid preserving compositions of the present invention are utilized in conjunction with a home specimen collecting kit, an individual can collect a specimen of biological fluid, such as blood, saliva, urine, and the like, seal the specimen in the inventive preserving composition in a container, and can ship the so-preserved specimen to a clinical laboratory for analysis of the specimen, without the need to visit a doctor's office, lab, or other out patient facility. The specimen can be analyzed after storage or transit for up to about 3 weeks at ambient room temperature without significantly affecting the concentration or detectability of protein analytes in the specimen.

The compositions of the present invention are particularly useful as a blood preserving medium for use in the Biosafe BTS™ blood collecting device. The BTS™ blood collecting device is designed to contain a blood preservation composition, and the device automatically meters in a specified amount of blood into the preservation fluid when the device is sealed. The device, containing the preserved blood sample, can then be shipped to a clinical laboratory, such as Biosafe Laboratories, of Lake Forest, Ill., for various clinical blood tests, such as a quantitative determination of TSH. The combination of the BTS™ blood collecting device with the biological fluid preserving compositions of the present invention affords a particularly effective means for achieving reliable home-collection of blood specimens for clinical blood analysis.

The following non-limiting examples are provided to further illustrate the biological fluid preserving compositions of the present invention and to illustrate the use of preferred inventive compositions to preserve biological fluid specimens in a real-world clinical environment.

Methods and Materials

TSH Analysis Preserved whole blood samples were analyzed by a modification of the Nichols Institute Diagnostics (NID) Third Generation Chemiluminescence Assay. According the manufacturer's product literature, the NID assay utilizes a mouse monoclonal antibody to TSH that is immobilized on a polystyrene (PS) bead, and a goat polyclonal antibody reagent labeled with a chemiluminescent acridinium ester. The goat antibody reagent is added to a serum sample in a test tube, and then the PS bead is added to the serum/goat antibody mixture. The PS bead binds and concentrates the TSH present in the serum, and the labeled goat antibody binds to the PS bead-bound TSH to form a “sandwich” complex with the TSH. The bead is then washed with saline to remove any remaining serum and non-bound goat antibody reagent. The washed bead is then placed in a luminometer and luminescence trigger reagents are added to cause the acridinium esters bound to the goat antibodies to luminesce. The TSH concentration is directly proportional to the luminescence signal in relative light units (RLU). The concentration of TSH in the serum sample is quantitatively determined by comparison of the sample RLU value with a calibration curve.

The assay was modified by Biosafe Laboratories, Inc. to adapt it for measurement of TSH in whole blood samples, rather than serum. The assay was adapted by using a smaller sample size (about 100 μL of stabilized whole blood versus about 200 μL of serum), dilution of the stabilized whole blood with a protein-rich diluent (about 150 μL), and use of only about 40 μL of goat antibody versus about 100 μL for the standard NID assay. Incubation time was also increased from about 2 hours for the standard assay to a time in the range of about 16 to about 20 hours in the modified assay. The standards and controls were also diluted to take into account the blood sample dilutions with the preservative solution and protein-rich diluent. The modified assay protocol is described in detail below.

A sample of about 100 μL of whole blood is diluted with the inventive preservative composition and is pipetted directly from the BTS™ blood collecting device to a borosilicate tube having a diameter of about 12 mm and a length of about 75 mm. A TSH goat polyclonal antibody is then added to the tube followed by a protein rich diluent (about 150 μL) and a NID TSH antibody PS bead. The tube containing the sample, antibody and PS bead is then incubated for about 16 to about 20 hours at about 22° C. The bead is then washed with isotonic saline using a NID System washer and the borosilicate tube, containing the washed bead, is placed in a NID Luminometer. The TSH concentration in the blood sample is then determined by comparing the observed chemiluminescence signal (in RLU) to a calibration curve. The analytical range of the procedure is in the range of about 0.04 to about 52.0 μIU/mL for TSH in whole blood. All reagents used in performing the analysis were obtained from Nichols Institute Diagnostics of San Juan Capistrano, Calif.

### EXAMPLE 1

### Biological Fluid Preserving Composition A

A biological fluid preserving composition was prepared by admixing the following ingredients: about 13 parts by weight of disodium EDTA, and about 0.22 parts by weight of sodium azide, were dissolved in about 437 parts by weight of deionized water; about 76 parts by weight of ethanol, and about 486 parts by weight of ethylene glycol were then added to the aqueous solution of EDTA and sodium azide with mixing agitation until a homogeneous solution was formed (Composition A).

Composition A remained stable and suitable for use as a biological fluid preservative for a period of over one year when stored at room temperature in sealed container.

### EXAMPLE 2

### Clinical Evaluation of Preservation of Home-Collected Whole Blood Samples

About 0.2 mL of Composition A from Example 1 was placed in the empty specimen holding chamber of a Biosafe BTS™ blood collecting device. A total of 186 such devices were prepared containing Composition A as the fluid preserving device. The devices were distributed to 186 volunteers who deposited blood samples into the devices by lancing a finger with a lancet (SURGILANCE®, available from SurgiLance Corp., Singapore) and squeezing about 3 drops of blood into the BTS™ blood collecting device collection port and sealing the port when the collection was complete. Each BTS™ blood collecting device automatically metered about 80 μL of blood into the Composition A in the specimen storage chamber of the device and the volunteers then mailed the devices containing the preserved blood samples to a clinical laboratory (Biosafe Laboratories, Inc.) for quantitative determination of TSH in the blood. The same volunteers also had blood samples collected by professional technicians for standard laboratory analysis for TSH. The analytical results for TSH level in the blood obtained with the BTS™ blood collecting device containing Composition A as a preservative were compared with analytical results for TSH obtained from the professionally drawn samples. The results are provided in Table 1.

As demonstrated by the data in Table 1, in-home collection of blood utilizing the BTS™ blood collecting device containing a biological fluid preserving composition of the present invention afforded analytical results for TSH concentrations that were essentially indistinguishable from results obtained utilizing professionally-collected blood samples.

TABLE 1

| | BTS ™ Device Collected | Prof. Collected |
|---|---|---|
| Mean TSH Concentration | 2.213 μIU/mL | 2.215 μIU/mL |
| Standard Deviation | 4.82 | 5.11 |

Total range 0.04-65.9 μIU/mL N = 186;
Correlation Coefficient 0.963; Slope 0.914, Intercept-0.04

### EXAMPLE 3

### Blood TSH Stability

In order to evaluate the effectiveness of the compositions of the present invention to preserve whole blood specimens for quantitative determination of TSH, several 80 μL samples of blood containing about 0.29 μIU/mL of TSH (to simulate a low level of TSH in a patient-collected-sample) were diluted in about 0.2 mL of Composition A per sample. The diluted blood samples were sealed in vials and stored at ambient temperatures of about 45° C., about 37° C., at an ambient room temperature of about 22° C. and at a refrigerated temperature in the range of about 2 to about 8° C. for about 21 days. One set of samples was stored at an ambient temperature that cycled from about −20° C. to about +37° C. on a daily basis for about 21 days. The TSH concentrations of the stored samples were determined at days 1, 3, 7, 14 and 21 (day zero being the day the samples were placed in storage). The results are presented in FIG. 1 and Table 2.

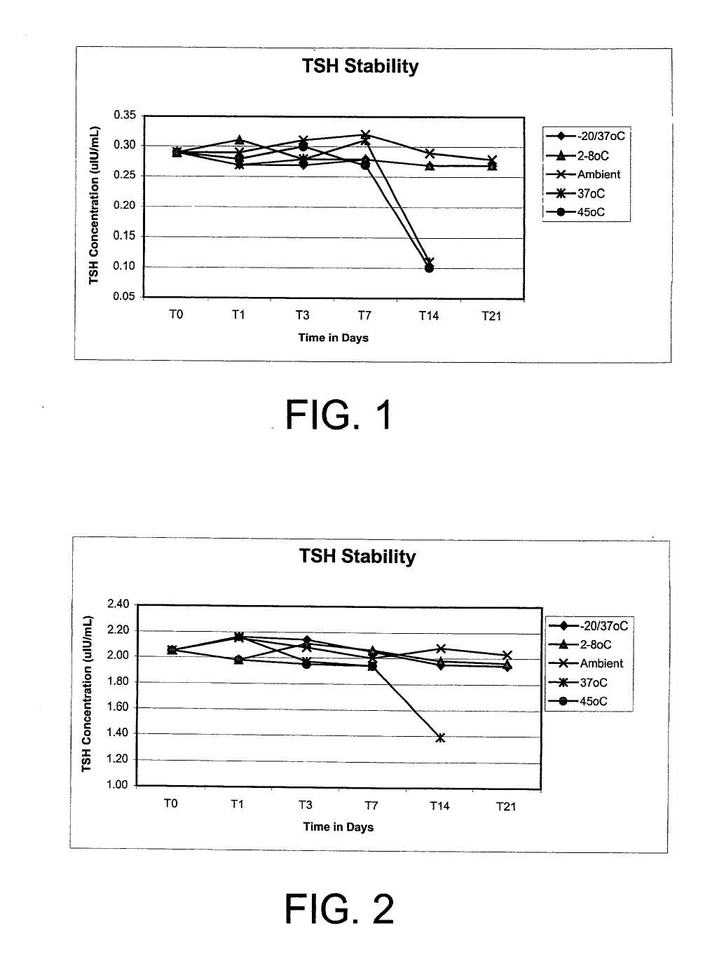

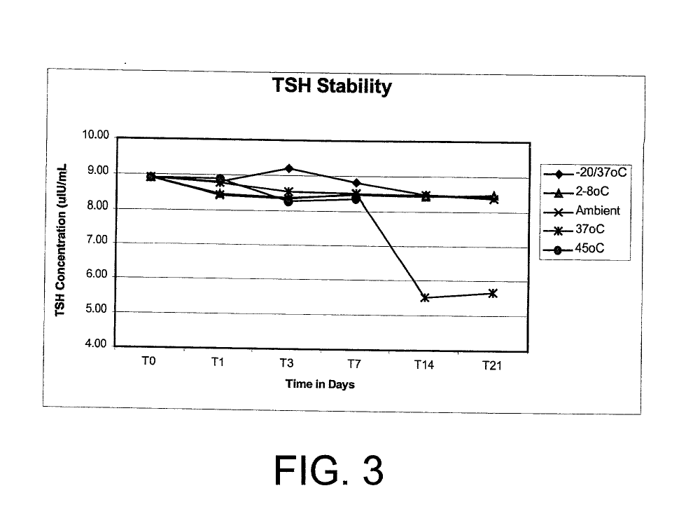

The stability test was repeated utilizing blood having TSH concentrations of about 2.05 μIU/mL (medium level of TSH in blood, results reported in Table 3 and FIG. 2) and about 8.91 μIU/mL (high level of TSH in blood, results reported in Table 4 and FIG. 3).

TABLE 2
Ave. TSH Concentration on Indicated Day, μIU/mL

| | Day 0 | Day 1 | Day 3 | Day 7 | Day 14 | Day 21 |
|---|---|---|---|---|---|---|
| 2 to 8° C. | 0.29 | 0.31 | 0.28 | 0.28 | 0.27 | 0.27 |
| 22° C. | 0.29 | 0.29 | 0.31 | 0.32 | 0.29 | 0.28 |
| 37° C. | 0.29 | 0.27 | 0.28 | 0.31 | 0.11 | QNS |
| 45° C. | 0.29 | 0.28 | 0.30 | 0.27 | 0.10 | QNS |
| −20 to +37° C. | 0.29 | 0.27 | 0.27 | 0.28 | 0.27 | 0.27 |

TABLE 3
Ave. TSH Concentration on Indicated Day, μIU/mL

| | Day 0 | Day 1 | Day 3 | Day 7 | Day 14 | Day 21 |
|---|---|---|---|---|---|---|
| 2 to 8° C. | 2.05 | 1.98 | 2.11 | 2.06 | 1.98 | 1.96 |
| 22° C. | 2.05 | 2.15 | 2.08 | 2.00 | 2.08 | 2.03 |
| 37° C. | 2.05 | 2.16 | 1.97 | 1.94 | 1.39 | QNS |
| 45° C. | 2.05 | 1.98 | 1.95 | 1.94 | QNS | QNS |
| −20 to +37° C. | 2.05 | 2.16 | 2.14 | 2.05 | 1.95 | 1.94 |

TABLE 4
Ave. TSH Concentration on Indicated Day, μIU/mL

| | Day 0 | Day 1 | Day 3 | Day 7 | Day 14 | Day 21 |
|---|---|---|---|---|---|---|
| 2 to 8° C. | 8.91 | 8.45 | 8.56 | 8.46 | 8.42 | 8.47 |
| 22° C. | 8.91 | 8.41 | 8.63 | 8.49 | 8.46 | 8.43 |
| 37° C. | 8.91 | 8.78 | 8.53 | 8.50 | 5.52 | 5.67 |
| 45° C. | 8.91 | 8.89 | 8.46 | 8.42 | QNS | QNS |
| −20 to +37° C. | 8.91 | 8.80 | 9.20 | 8.82 | 8.47 | 8.41 |

In the tables QNS=quantity not sufficient for measurement.

The data in Tables 2-4 and FIGS. 1-3 demonstrate that the inventive biological fluid preserving Composition A can stabilize a protein analyte, such as TSH, in a whole blood sample for up to about 3 weeks with no significant loss of TSH activity when stored at ambient temperatures at or below about 22° C. Similarly, samples stored at ambient temperatures that cycled between about −20° C. to about +37° C. daily were also stable for up to about 3 weeks. TSH containing blood samples stored at about 37° C. and about 45° C. were stable for up to about 1 week when preserved in Composition A.

Numerous variations and modifications of the embodiments described above may be effected without departing from the spirit and scope of the novel features of the invention. It is to be understood that no limitations with respect to the specific embodiments illustrated herein are intended or should be inferred. It is, of course, intended to cover by the appended claims all such modifications as fall within the scope of the claims.

We claim:

1. An aqueous biological fluid preserving composition suitable for preservation of a biological fluid specimen consisting essentially of a chelating agent and a cell lysing amount of a cell lysing agent in water.

2. The composition of claim 1 wherein the chelating agent is a calcium chelating agent.

3. The composition of claim 1 wherein the chelating agent is EDTA.

4. The composition of claim 1 wherein the cell lysing agent is a C₁-C₄ alcohol.

5. The composition of claim 4 wherein the cell lysing agent is ethanol.

6. The composition of claim 1 wherein the aqueous biological fluid preserving composition further includes a preservative.

7. The composition of claim 6 wherein the preservative is sodium azide.

8. The composition of claim 1 wherein the aqueous biological fluid preserving composition further includes an antifreeze agent.

9. The composition of claim 8 wherein the antifreeze agent is an organic polyol.

10. The composition of claim 9 wherein the organic polyol is a C₁-C₁₀ polyol.

11. The composition of claim 8 wherein the antifreeze agent is ethylene glycol.

12. An aqueous biological fluid preserving composition suitable for preservation of a biological fluid specimen consisting essentially of:
a) about 0.05 to about 0.5 weight percent of a chelating agent;
b) about 5 to about 25 weight percent of a cell lysing agent;
c) up to about 0.1 weight percent of a preservative; and
d) the remainder being water.

13. The composition of claim 12 wherein the chelating agent is a calcium chelating agent.

14. The composition of claim 12 wherein the chelating agent is EDTA.

15. The composition of claim 12 wherein the cell lysing agent is a C₁- C₄ alcohol.

16. The composition of claim 15 wherein the cell lysing agent is ethanol.

17. The composition of claim 12 wherein the preservative is present in an amount in the range of about 0.01 to about 0.03 weight percent.

18. The composition of claim 17 wherein the preservative is sodium azide.

19. The composition of claim 12 wherein the aqueous biological fluid preserving composition further includes up to about 50 weight percent of an antifreeze agent.

20. The composition of claim 19 wherein the antifreeze agent is an organic polyol.

21. The composition of claim 20 wherein the organic polyol is a C₁- C₁₀ polyol.

22. The composition of claim 19 wherein the antifreeze agent is ethylene glycol.

23. A method of preserving a protein-containing biological fluid specimen comprising the sequential steps of:
a) providing a sealable container, suitable for storing or transporting a biological fluid sample;
b) combining in said container a biological fluid specimen and an aqueous biological fluid preserving solution consisting essentially of a chelating agent and a cell lysing amount of a cell lysing agent in water; and
c) sealing the container.

24. The method of claim 23 wherein the biological fluid specimen is admixed with about 0.5 to about 8 volumes of the aqueous preservative solution, based on the volume of the specimen.

25. The method of claim 23 wherein the chelating agent is a calcium chelating agent.

26. The method of claim 23 wherein the cell lysing agent is a C₁- C₄ alcohol.

27. The method of claim 23 wherein the aqueous biological fluid preserving solution further includes a preservative.

28. The method of claim 27 wherein the preservative is sodium azide.

29. The method of claim 23 wherein the aqueous biological fluid preserving solution further includes an antifreeze agent.

30. The method of claim 29 wherein the antifreeze agent is a C₁- C₁₀ polyol.

31. An article of manufacture comprising an aqueous biological fluid preserving composition of claim 1 in packaged form.

32. An article of manufacture comprising an aqueous biological fluid preserving composition of claim 12 in packaged form.

33. A biological fluid test kit comprising a sealable biological fluid collecting receptacle containing an aqueous biological fluid preserving composition of claim 1, packaged with instructional materials describing how to collect the biological specimen and instructions for delivery of the collected specimen to a laboratory for analysis.

34. A biological fluid test kit comprising a sealable biological fluid collecting receptacle containing a biological fluid preserving composition of claim 12, packaged with instructional materials describing how to collect the biological specimen and instructions for delivery of the collected specimen to a laboratory for analysis.
